SEQUENCE LISTING (18 sequences)

SEQ ID NO: 1 - protein, 452 aa, Artificial
  Cetuximab H-chain
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, Artificial
  Cetuximab L-chain
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGA

SEQ ID NO: 3 - protein, 6 aa, Artificial
  Cetuximab signature peptide
SQVFFK

SEQ ID NO: 4 - protein, 451 aa, Artificial
  Rituximab H-chain
QVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 213 aa, Artificial
  Rituximab L-chain
QIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 20 aa, Artificial
  Rituximab signature peptide 1
GLEWIGAIYPGNGDTSYNQK

SEQ ID NO: 7 - protein, 447 aa, Artificial
  Brentuximab vedotin H-chain
QIQLQQSGPEVVKPGASVKISCKASGYTFTDYYITWVKQKPGQGLEWIGWIYPGSGNTKYNEKFKGKATLTVDTSSSTAFMQLSSLTSEDTAVYFCANYGNYWFAYWGQGTQVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 218 aa, Artificial
  Brentuximab vedotin L-chain
DIVLTQSPASLAVSLGQRATISCKASQSVDFDGDSYMNWYQQKPGQPPKVLIYAASNLESGIPARFSGSGSGTDFTLNIHPVEEEDAATYYCQQSNEDPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 16 aa, Artificial
  Brentuximab vedotin signature peptide
VLIYAASNLESGIPAR

SEQ ID NO: 10 - protein, 9 aa, Artificial
  Trastuzumab signature peptide
IYPTNGYTR

SEQ ID NO: 11 - protein, 9 aa, Artificial
  Bevacizumab signature peptide
FTFSLDTSK

SEQ ID NO: 12 - protein, 15 aa, Artificial
  Rituximab signature peptide 2
ASGYTFTSYNMHWVK

SEQ ID NO: 13 - protein, 15 aa, Artificial
  Nivolumab signature peptide
ASGITFSNSGMHWVR

SEQ ID NO: 14 - protein, 22 aa, Artificial
  Ipilimumab signature peptide
ASQSVGSSYLAWYQQKPGQAPR

SEQ ID NO: 15 - protein, 11 aa, Artificial
  Ramucirumab signature peptide
AFPPTFGGGTK

SEQ ID NO: 16 - protein, 13 aa, Artificial
  Infliximab signature peptide 1
SINSATHYAESVK

SEQ ID NO: 17 - protein, 15 aa, Artificial
  Infliximab signature peptide 2
ASQFVGSSIHWYQQR

SEQ ID NO: 18 - protein, 10 aa, Artificial
  Adalimumab signature peptide
APYTFGQGTK

(19) United States
(12) Patent Application Publication
(10) Pub. No.: US 2020/0011876 A1
(43) Pub. Date: Jan. 9, 2020

(54) Method for Simultaneous Quantification of Monoclonal Antibodies

(71) Applicant: Shimadzu Corporation, Kyoto (JP)

(72) Inventors: Noriko Iwamoto, Kyoto-shi, Kyoto (JP); Megumi Takanashi, Kyoto-shi, Kyoto (JP); Takashi Shimada, Kyoto-shi, Kyoto (JP)

(73) Assignee: Shimadzu Corporation, Kyoto (JP)

(21) Appl. No.: 16/493,544

(22) PCT Filed: Mar. 14, 2017
(86) PCT No.: PCT/JP2017/010232

§ 371 Date: Sep. 12, 2019

(51) Int. Cl. G01N 33/68; G01N 33/577

(52) U.S. Cl. CPC G01N 33/6848; G01N 33/577

(57) ABSTRACT

The present invention provides a technique that can simultaneously analyze a plurality of antibody drugs and can be validated. The present invention provides a method of bringing a porous body in which monoclonal antibodies to be measured are immobilized in pores into contact in a liquid with nanoparticles on which proteases are immobilized and performing selective proteolysis of monoclonal antibodies to detect peptide fragments each comprising unique amino acid sequence derived from the Fab region of the monoclonal antibodies via liquid chromatography-mass spectrometry (LC-MS), wherein peptide fragments of two or more types of monoclonal antibodies in the same biological sample are simultaneously quantified.

### TECHNICAL FIELD

The present invention relates to a method for quantification of monoclonal antibodies. More specifically, the present invention relates to a method for simultaneous detection and quantification of a plurality of antibodies that are concurrently present in a sample via mass spectrometry without separating such plurality of antibodies from one another.

### TECHNICAL BACKGROUND

In the past, protein components in organisms had been detected and quantified primarily via ligand binding assays (LBAs) (Non-Patent Documents 1 and 2). According to this technique, an antibody that specifically binds to a target protein as an antigen is prepared, and the antigen is then detected using a secondary antibody for detection that recognizes the antibody and a label, such as a fluorescent, chemoluminescent, lanthanide, spin label, or radioisotope label. A technique for preparing an antibody capable of binding to a particular antigen has been remarkably advanced. With the use of a polyclonal antibody or monoclonal antibody for relevant purposes, LBA has been extensively applied to research and development.

LBA can be used for an extensive range of applications, and analysis involving the use of, for example, a microtiter plate, is suitable for automation. Accordingly, LBA has still been extensively employed at present in spite of the fact that more than 50 years have passed since development of such technique.

In recent years, many monoclonal antibodies that bind to pathogenic proteins have been developed as molecular-targeted drugs used for treatment of cancers and autoimmune diseases, and used in clinical settings. Since monoclonal antibodies have very high molecular specificity, different monoclonal antibodies would be employed depending on diseases types and antigen recognition sites of target proteins. In order to perform the optimal medical treatment with the use of monoclonal antibodies, it has become necessary to quantify the concentration of the monoclonal antibodies in vivo after the administration thereof to the patients.

The present inventors have attempted to obtain peptides peculiar to relevant monoclonal antibodies in order to perform specific detection and quantification of monoclonal antibodies via mass spectrometry. As a result, they have succeeded in proteolysis through a position selective solid phase-solid phase reaction of monoclonal antibodies by immobilizing both monoclonal antibodies and proteases that can recognize such antibodies as substrates and digest the same (Patent Document 1 and Non-Patent Document 3). In this method, a porous body in which monoclonal antibodies to be measured are immobilized in pores is brought into contact with nanoparticles on which proteases are immobilized in a liquid to perform selective proteolysis of monoclonal antibodies, and resulting peptide fragments are detected effectively via liquid chromatography-mass spectrometry (LC-MS).

### RELATED ART

### Patent Documents

[Patent Document 1] WO 2015/033479

### Non-Patent Documents

[Non-Patent Document 1] J. Clin. Invest., 35, 170-190, 1956
[Non-Patent Document 2] J. Clin. Invest., 38, 1996-2016, 1959
[Non-Patent Document 3] Analyst., Feb. 7 2014; 139 (3): 576-80, DOI: 10.1039/c3an02104a

### SUMMARY OF THE INVENTION

### Problems to be Solved by the Invention

In order to perform detection and quantification of monoclonal antibodies in a biological sample, the LBA technique has technical problems as described below.
(i) Antibody production requires a period of 6 to 10 months and a cost of approximately 5,000,000 Japanese yen.
(ii) Whether or not the antibody would actually recognize an antigen of interest should be examined through the final screening.
(iii) The LBA technique is directly affected by a coexisting biological matrix (e.g., blood, a cell extract, a host animal, an allergen, or an autoantibody) and a reagent such as a surfactant.
(iv) Verification is difficult because an antigen (a monoclonal antibody medicine) is not directly detected.
(v) The reference calibration curve requires characteristic fitting. Accordingly, a variation in concentration would be significant at the lower limit of quantification and at the upper limit of quantification.
(vi) Detection of a plurality of antigens requires the use of a plurality of antibodies and relevant secondary antibodies for dedicated purposes.

In recent years, combination chemotherapy involving the use of a plurality of medicines has advanced in the field of, in particular, cancer therapy, and simultaneous use of a variety of antibody drugs has become practical. For example, clinical trials have been conducted concerning combination therapy for melanoma involving the use of an anti-PD-1 antibody Nivolumab, an anti-CTLA-4 antibody Ipilimumab, and an anti-VGEF-a antibody Bevacizumab. Under such circumstances, it is necessary to monitor pharmacokinetics of antibody drugs in a simple manner with certainty. Accordingly, it is highly unlikely that LBA that would be affected by the matrix and would incur high costs be sufficient. In particular, an extensive range of applications of a technique that can perform simultaneous analysis of a plurality of antibody drugs and can be validated would be expected in the future.

In order to detect and quantify a protein via mass spectrometry in a simple manner, it is necessary to efficiently obtain a peptide fragment specific for a protein to be measured.

### Means for Solving the Problems

Under the above circumstances, the present inventors have attempted quantification of a variety of monoclonal antibodies by employing, as a pretreatment technique for mass spectrometry of monoclonal antibodies, a method of nano-surface and molecular-orientation limited proteolysis (hereafter, referred to as the “nSMOL” method) in which monoclonal antibodies are subjected to selective proteolysis through the solid phase-solid phase reaction, developed by the present inventors in the past. It has been demonstrated that the method of analysis via the nSMOL method is capable of detecting monoclonal antibodies existing in a biological sample with high sensitivity and selectivity, concerning a plurality of monoclonal antibodies.

The present inventors discovered that two or more types of monoclonal antibodies existing in a biological sample could be concurrently and simultaneously detected and quantified by the nSMOL method. In addition, the method of the present invention involving the nSMOL method was found to satisfy the standards of the guideline on bioanalytical method validation in Japan, U.S.A., and Europe.

Specifically, the present invention provides the following.
1. A method for detecting a peptide fragment having the amino acid sequence derived from the Fab region of a monoclonal antibody comprising bringing a porous body in which monoclonal antibodies to be measured are immobilized in pores into contact with nanoparticles on which proteases are immobilized in a liquid to perform selective proteolysis of the monoclonal antibodies via liquid chromatography-mass spectrometry (LC-MS), wherein peptide fragments of two or more types of monoclonal antibodies in the same biological sample are simultaneously quantified.
2. The method according to 1. above, wherein concentration of each of the two or more types of monoclonal antibodies in a biological sample is 0.5 to 300 μg/ml.
3. The method according to 1. or 2. above, wherein the results of simultaneous quantification of the two or more types of monoclonal antibodies exhibit accuracy of ±15% derivation from the results attained when the monoclonal antibodies are quantified independently of each other.
4. The method according to any of 1. to 3. above, wherein 3, 4, 5, 6, 7, 8, 9, 10, or more types of monoclonal antibodies are simultaneously quantified.
5. The method according to any of 1. to 4. above, wherein the monoclonal antibodies include an antibody-drug complex.
6. The method according to any of 1. to 5. above, wherein the monoclonal antibodies include two or more types of antibodies selected from among: human antibodies such as Panitumumab, Ofatumumab, Golimumab, Ipilimumab, Nivolumab, Ramucirumab, and Adalimumab; humanized antibodies such as Tocilizumab, Trastuzumab, Trastuzumab-DM1, Bevacizumab, Omalizumab, Mepolizumab, Gemtuzumab, Palivizumab, Ranibizumab, Certolizumab, Ocrelizumab, Mogamulizumab, and Eculizumab; chimeric antibodies such as Rituximab, Cetuximab, Infliximab, Basiliximab, Brentuximab vedotin, and Gemtuzumab ozogamicin; and an antibody-drug complex such as Trastuzumab emtansine.
7. The method according to 6. above, wherein the monoclonal antibodies are two or three types of antibodies selected from among Cetuximab, Rituximab, and Brentuximab vedotin.
8. A composition used for combined quantification of monoclonal antibodies in a biological sample via liquid chromatography-mass spectrometry (LC-MS), which comprises two or more types of peptide fragments obtained by selective proteolysis, each comprising an amino acid sequence derived from the Fab region of a monoclonal antibody.
9. The composition according to 8. above, which yields stable results of quantification at 5° C. for 48 hours after selective proteolysis.
10. The composition according to 8. or 9. above, wherein the monoclonal antibodies include Cetuximab and a peptide fragment to be measured has the amino acid sequence as shown in SEQ ID NO: 3.
11. The composition according to 8. or 9. above, wherein the monoclonal antibodies include Rituximab and a peptide fragment to be measured has the amino acid sequence as shown in SEQ ID NO: 6.
12. The composition according to 8. or 9. above, wherein the monoclonal antibodies include Brentuximab vedotin and a peptide fragment to be measured has the amino acid sequence as shown in SEQ ID NO: 9.

### Effects of the Invention

The method of analysis involving the nSMOL method according to the present invention was found to be capable of detecting monoclonal antibodies existing alone or in combinations of two or more in a biological sample with high sensitivity and accuracy. The method of the present invention enables simultaneous measurement of a plurality of antibody drugs.

According to the method of the present invention, a plurality of antibodies can be simultaneously analyzed without preparing antibodies that specifically bind to and detect relevant antibody drugs, and concentrations of antibodies in vivo can be monitored. Thus, the cost and the time required for development of analytical techniques in clinical settings can be reduced to a significant extent, and complicated pharmacokinetic information concerning antibody drugs can be readily provided.

### BRIEF DESCRIPTION OF THE DRAWINGS

FIG. 1 schematically shows the nSMOL method.

FIG. 2 shows calibration curves prepared based on combined quantification of standard samples containing Cetuximab, Rituximab, and Brentuximab vedotin.

FIG. 3 shows the results of MRM assays of signature peptides following the pretreatment via the nSMOL method of a plasma sample containing all of Trastuzumab, Bevacizumab, Cetuximab, Rituximab, Nivolumab, Ipilimumab, Ramucirumab, Brentuximab vedotin, Infliximab, and Adalimumab at 10 μg/ml each and samples each containing one of the monoclonal antibodies at 10 μg/ml. The results of combined quantification are demonstrated in the form of an ion yield relative to the ion yield attained by independent quantification designated to be 100.

### EMBODIMENTS FOR CARRYING OUT THE INVENTION

In one embodiment, the present invention relates to a method for detecting a peptide fragment comprising an amino acid sequence derived from the Fab region of a monoclonal antibody, comprising bringing a porous body in which monoclonal antibodies to be measured are immobilized in pores into contact in a liquid with nanoparticles on which proteases are immobilized to perform selective proteolysis of the monoclonal antibodies, via liquid chromatography-mass spectrometry (LC-MS), wherein peptide fragments of two or more types of monoclonal antibodies in the same biological sample are simultaneously quantified. Types of monoclonal antibodies that can be simultaneously quantified are 3, 4, 5, 6, 7, 8, 9, 10, or more. In the method of the present invention, surprisingly, the present inventors simultaneously quantified 12 types of peptides derived from 10 types of monoclonal antibodies and verified that the results of quantification were not influenced.

The term “biological sample” used herein refers to, in a clinical sense, a sample derived from the blood or tissue of a patient to which monoclonal antibodies had been administered in the form of antibody drugs. The term preferably refers to a plasma, serum, or tissue homogenate extract. A biological sample can be subjected to the method of the present invention immediately after it is obtained from a patient or subject. Alternatively, the sample may be stored at room temperature or low temperature and then subjected to the method of the present invention.

In the method of the present invention, concentration of each of two or more types of monoclonal antibodies in a biological sample may be within a range of 0.5 to 300 μg/ml. At such concentration, sensitivity and accuracy are very high.

The method of the present invention can yield very stable results of quantification under various conditions. For example, the results of simultaneous quantification of two or more types of monoclonal antibodies exhibit accuracy of ±15% deviation from the results attained when each of such monoclonal antibodies are quantified independently. The present inventors verified that the method of the present invention would provide highly accurate results of detection with good reproducibility even after storage at room temperature for a short period of time (i.e., 4 hours) and after cryopreservation at −20° C. or −80° C. for 20 to 30 days. In addition, the results of detection were not influenced when a biological sample was repeatedly subjected to freezing at −20° C. or −80° C. and thawing.

In another embodiment, the present invention provides a composition used for combined quantification of monoclonal antibodies in a biological sample via liquid chromatography-mass spectrometry (LC-MS), containing two or more types of peptide fragments each comprising an amino acid sequence derived from the Fab region of a monoclonal antibody obtained by selective proteolysis. This composition can be used as a standard material for simultaneous quantification of two or more types of monoclonal antibodies.

This composition is verified to be very stable. For example, the composition can provide stable quantification results at 5° C. for 48 hours after selective proteolysis. Specifically, peptide fragments obtained after selective proteolysis of monoclonal antibodies are stable in solution such as a buffer. For example, the results of detection attained after storage at 5° C. for 24 or 48 hours satisfy the standards of the guidelines described below, and the results of detection with high sensitivity can be obtained.

In order to detect and quantify monoclonal antibodies via mass spectrometry, it is necessary to first eliminate substances other than the material to be measured from the biological sample such as blood or tissue as much as possible and then dissolve the sample in an adequate solvent. In addition, the molecular weight of an antibody is too large to be analyzed in that state. Thus, the antibody is degraded into a peptide with the aid of a protease, separated via liquid chromatography, and then subjected to mass spectrometry. The molecular weight of peptides suitable for analysis is approximately 1000 to 3000 Da.

When a common protein molecule is degraded with a protease, however, about 100 peptide fragments are generated. In the case of an antibody, the number of peptide fragments generated exceeds 200 to a significant extent. Accordingly, the number of targets to be measured is numerous even for a single protein, and enormous number of samples should be analyzed when complicated biological samples are the targets.

As the number of peptides to be analyzed increases, it is difficult to completely separate peptides from each other via column separation. This would lower the ionization efficiency attained by matrix effects, and sensitivity and quantification reproducibility would be lowered as a consequence. In order to overcome such problems, high-performance channel switching functions is created for mass spectrometry. However, such matrix effects cannot be overcome without reducing the general population.

The nSMOL method developed by the present inventors can be used as a method of pretreatment for mass spectrometry that generates Fab region-selective peptide fragments effective for detection of monoclonal antibodies.

Concerning methods for analyzing drug concentration in biological samples, similar validation guidelines have been published in Japan, U.S.A., and Europe: i.e., “Guideline on Validation of Analytical Methods for Drug Concentration in Biological Samples in Pharmaceutical Development”, PFSB/ELD Notification No. 0711-1, 2013, Ministry of Health, Labour and Welfare, Japan; “Guidance for Industry, Bioanalytical Method Validation”, U.S. Food and Drug Administration (FDA), 2013; and “Guideline on bioanalytical method validation”, European Medicines Agency (EMA), 2011.

In summary, major items of the guidelines published by the Evaluation Division, the Pharmaceutical and Food Safety Bureau, the Ministry of Health, Labour and Welfare are as described below.

Selection of internal standard material: The material with certified quality should be selected, and such material should not affect analysis of the material to be analyzed.

Analysis selectively: Analysis should not be adversely affected by blank samples obtained from 6 individuals (male: 3; female: 3).

Lower limit of quantification (LLOQ): The analyte response at lower limit of quantification; the lowest concentration at which the analyte can be quantified with reliable accuracy and precision should be at least 5 times the response of that in a blank sample, and mean accuracy should be within ±20% deviation from the nominal concentration.

Calibration curve: The mean accuracy of samples of at least 6 concentration levels including LLOQ should be within ±20% deviation at the LLOQ and within ±15% deviation at all other levels.

Accuracy and precision: Accuracy and precision for QC samples with 4 different concentrations (LLOQ and low-, mid-, and high-levels) should be within ±20% deviation at the LLOQ and within ±15% deviation at all other levels.

Matrix effect: The precision of the matrix factor evaluating the influence on the analyte in a biological sample is within 15% among individuals and the precision of quantified values for QC samples prepared with the use of matrices obtained from 6 individuals is within 15% among individuals.

Carry-over: The response in the blank sample obtained after analysis of the sample at the highest concentration should not be greater than 20% of the response at the LLOQ.

Dilution integrity: Mean accuracy in the measurements of the sample diluted from the level outside the range of quantification using the calibration curve should be within ±15% deviation from the theoretical concentration.

Sample stability: When samples at low and high concentrations are evaluated for their stability by being allowed to stand at room temperature for 4 hours, by being subjected to freeze and thaw cycles 5 times, for long-term storage stability of approximately 1 month, and for stability 1 day and 2 days after sample treatment, the mean accuracy at each concentration should be within ±15% deviation from the theoretical concentration.

The present inventors selected Cetuximab, Rituximab, and Brentuximab vedotin as examples of monoclonal antibodies that can be quantified in the present invention. Samples containing each antibody in the plasma were independently quantified by the method of the present invention including the nSMOL method, the calibration curves in the plasma were prepared, and analysis results were fully validated in accordance with the guideline on validation of analytical methods for drug concentration in biological samples in pharmaceutical development, PFSB/ELD Notification No. 0711-1, 2013, the Ministry of Health, Labour and Welfare, Japan.

Subsequently, whether or not similar calibration curves could be obtained from plasma samples containing the 3 types of monoclonal antibodies was examined under the same analytical conditions as with the independent quantification described above.

As a result, the method of the present invention was verified to satisfy the standards of the guidelines and provide the quantification results with high accuracy and high sensitivity. In addition, the method of the present invention was verified to provide stable quantification results under various conditions.

<Summary of the nSMOL Method>

The method of the present invention is implemented by adopting the nSMOL method developed by the present inventors in the past. The nSMOL method is described in detail in, for example, WO 2015/033479 and Iwamoto, N. et. al., Selective detection of complementarity-determining regions of monoclonal antibody by limiting protease access to the substrate: nano-surface and molecular-orientation limited proteolysis, Analyst., Feb. 7 2014; 139 (3): 576-80, DOI: 10.1039/c3an02104a. In addition, improved techniques of the nSMOL method are disclosed in, for example, WO 2016/143223, WO 2016/143224, WO 2016/143226, WO 2016/143227, Iwamoto, N. et. al., Bioanalysis, doi: 10.4155/bio-2016-0018, and Iwamoto, N. et. al., Biological & Pharmaceutical Bulletin, 2016, doi:10.1248/bpb.b16-00230. The contents of the documents indicated above are incorporated herein by reference.

More specifically, the nSMOL method comprises bringing a porous body in which monoclonal antibodies to be measured are immobilized in pores into contact in a liquid with nanoparticles on which proteases are immobilized and performing selective proteolysis of monoclonal antibodies. Peptides obtained by the nSMOL method preferably comprise an amino acid sequence derived from an antibody Fab region, such as amino acids derived from the heavy chain or light chain CDR2 region.

### <Antibody>

A monoclonal antibody to be measured in the method of the present invention is an immunoglobulin (IgG) in which the Fab domain and the Fc domain are connected via a hinge, and two heavy chains and two light chains constituting the antibody molecule are respectively formed of a constant region and a variable region. The constant region has an amino acid sequence that is common to most of antibodies derived from the same species. On the other hand, the variable region has three sites each having a specific sequence called a complementarity determining region (CDR). The three-dimensional structure defined by these CDR (CDR1, CDR2, and CDR3) regions is involved in specific binding with an antigen, and thereby, an antibody-antigen complex is formed.

Examples of monoclonal antibodies to be measured in the method of the present invention include, but are not limited to: human antibodies such as Panitumumab, Ofatumumab, Golimumab, Ipilimumab, Nivolumab, Ramucirumab, and Adalimumab; humanized antibodies such as Tocilizumab, Trastuzumab, Trastuzumab-DM1, Bevacizumab, Omalizumab, Mepolizumab, Gemtuzumab, Palivizumab, Ranibizumab, Certolizumab, Ocrelizumab, Mogamulizumab, and Eculizumab; and chimeric antibodies such as Rituximab, Cetuximab, Infliximab, and Basiliximab. The molecular diameter of monoclonal antibodies is about 14.5 nm.

Further, a complex having an additional function while maintaining specificity of a monoclonal antibody, such as an Fc fusion protein, an antibody-drug complex (such as Brentuximab vedotin, Gemtuzumab ozogamicin, and Trastuzumab emtansine) is also included in monoclonal antibodies to be measured in the method of the present invention. Prior to measurement, binding of the complex may be dissociated, and only the antibody may be subjected to analysis. Alternatively, the complex itself may be subjected to analysis. As described in the examples, the present inventors succeeded in subjecting Brentuximab vedotin in the plasma to the nSMOL method in that state and then performing mass spectrometry following proteolysis. A person skilled in the art can set an optimal condition for the method of the present invention in accordance with a measurement target, based on the description of this specification.

The method of the present invention that employs the nSMOL method comprises subjecting the Fab region of a monoclonal antibody to selective proteolysis to obtain a peptide fragment, and subjecting the peptide fragment to mass spectrometry, thereby directly measuring the antibody-derived peptide fragment. Accordingly, the method of the present invention is applicable regardless of antibody type, and it is also applicable to newly developed monoclonal antibodies and the like.

### <Porous Body>

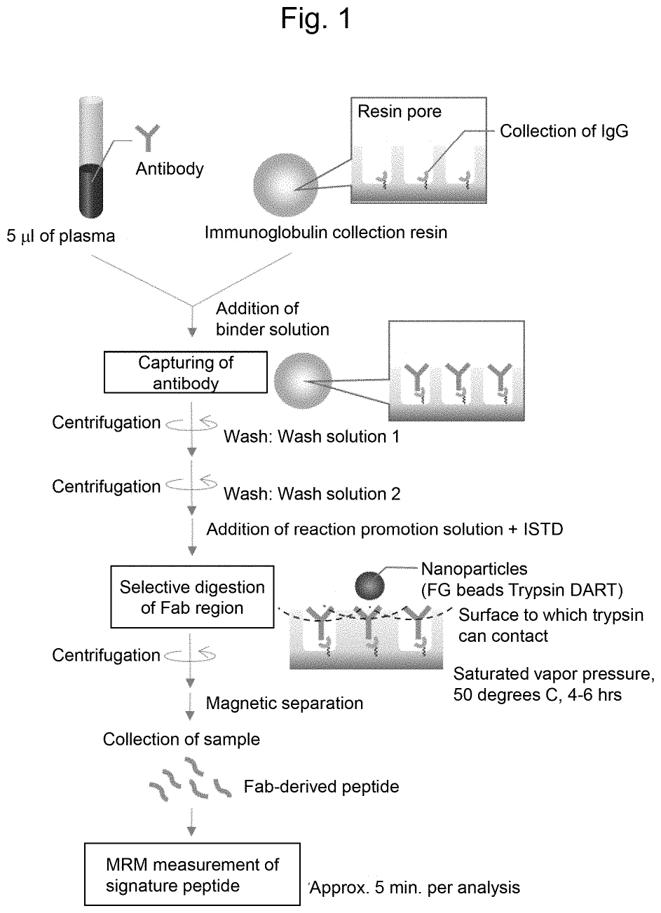

A material constituting a porous body used in the method of the present invention (“immunoglobulin collection resin” in FIG. 1) is not particularly limited as long as the porous body has a large number of pores, and activated carbon, a porous membrane, porous resin beads, metal particles, and the like can be used. Among these, those capable of site-specifically binding to an antibody are particularly preferable.

The pores are not particularly limited in shape. As with the case of a porous membrane, the porous body having pores formed to penetrate though the porous body can also be used. The size of a pore of the porous body is not particularly limited, and it is preferably determined by taking a molecular diameter of an antibody and the like into consideration, so that, when an antibody is immobilized, a site to be selectively digested would be positioned near the surface layer of the pore. The average pore size of the porous body is appropriately set in a range of about 10 nm to 200 nm and in the range smaller than the average particle size of nanoparticles. The average pore size of the porous body is, for example, preferably about 20 nm to 200 nm, and more preferably about 30 nm to 150 nm. In order to immobilize the Fc domain of an antibody in a pore and subject the Fab domain to position selective proteolysis, a pore size of a porous body is preferably 30 nm to 150 nm, more preferably 40 nm to 120 nm, even more preferably 50 nm to 100 nm, and particularly preferably about 100 nm.

In the nSMOL method, monoclonal antibodies to be measured are immobilized in pores of the porous body. To this end, a porous body in which linker molecules that site-specifically interact with antibodies are immobilized in pores is preferably used. Examples of interactions between antibodies and linker molecules include chemical bonding, hydrogen bonding, ionic bonding, complex formation, hydrophobic interaction, van der Waals interaction, electrostatic interaction, and stereoselective interaction.

As linker molecules, Protein A, Protein G, and the like that site-specifically bind to the Fc domain of an antibody are preferably used. With the use of a porous body in which these linker molecules are immobilized in pores, the Fc domain of an antibody is immobilized in a pore, and the Fab domain is positioned near a surface layer of the pore. By controlling orientation of an antibody in a pore, position selective digestion of the Fab domain by a protease becomes possible.

The size of a linker molecule is selected in a manner such that a selective cleavage site of an antibody is positioned near a surface layer of a pore. When a linker molecule is bound to an antibody, the molecular size in that state is preferably about 0.5 times to 1.5 times, more preferably about 0.6 times to 1.2 times, even more preferably about 0.7 times to 1.1 times, and particularly preferably about 0.8 times to 1 times the pore size of the porous body. When a linker molecule is not immobilized on a porous body and an antibody is directly bound in a pore, it is preferable that the molecular diameter of the antibody and the pore size of the porous body satisfy the above relation.

A porous body that can be suitably used in the present invention is not particularly limited. For example, Protein G Ultralink resin (manufactured by Pierce Corporation), Toyopearl TSKgel (manufactured by TOSOH Inc.), and Toyopearl AF-rProtein A HC-650F resin (manufactured by TOSOH Inc.) can be used.

A method for immobilizing an antibody in a pore of the porous body is not particularly limited. An appropriate method can be adopted in accordance with characteristics of an antibody, a porous body, a linker molecule, and the like. When an antibody is immobilized in a porous body in which protein A or protein G is immobilized in a pore, for example, a suspension of a porous body may be mixed with a solution containing an antibody. Thus, an antibody can be easily immobilized in a pore.

The quantitative ratio of a porous body to an antibody can be appropriately set according to a purpose. When an antibody is quantitatively analyzed, for example, it is desirable that substantially the entire amount of antibodies in the sample be immobilized in the porous body. Therefore, it is preferable that the quantitative ratio be set such that an amount of the porous body becomes excessive with respect to an estimated content of the antibodies in the sample.

### <Nanoparticles>

Nanoparticles are used to immobilize proteases on the nanoparticle surface and to control access of proteases to antibodies immobilized in pores of a porous body. Therefore, the average particle size of nanoparticles should be larger than the average pore size of the porous body, so that the nanoparticles would not enter deep into the pores of the porous body.

Nanoparticles are not particularly limited in shape. From the viewpoint of equalization of access of proteases to pores of the porous body, spherical nanoparticles are preferable. In addition, it is preferable that nanoparticles have high dispersibility and a uniform particle size.

A material of nanoparticles is not particularly limited as long as the proteases can be immobilized on the nanoparticle surface, and a metal, resin, or the like can be appropriately used. A metal coated with a resin, a resin coated with a metal, or the like can also be used.

As a type of the nanoparticles, magnetic nanoparticles that can be dispersed or suspended in an aqueous medium and can be easily recovered from the dispersion or suspension by magnetic separation or magnetic precipitation separation are preferable. From the viewpoint that aggregation is less likely to occur, magnetic nanoparticles covered with an organic polymer are more preferable. Examples of base materials of magnetic nanoparticles include ferromagnetic alloys such as iron oxide (magnetite (Fe₃O₄), maghemite (γ-Fe₂O₃)), and ferrite (Fe/M)₃O₄. In the ferrite (Fe/M)₃O₄, M means a metal ion that can be used in combination with an iron ion to form a magnetic metal oxide, and Co²⁺, Ni²⁺, Mn²⁺, Mg²⁺, Cu²⁺, Ni²⁺ and the like are typically used. Further, examples of the organic polymer covering magnetic nanoparticles include polyglycidyl methacrylate (poly GMA), a copolymer of GMA and styrene, polymethyl methacrylate (PMMA), and polymethyl acrylate (PMA). Specific examples of magnetic nanobeads coated with an organic polymer include FG beads, SG beads, Adembeads, and nanomag. As a commercially available product, for example, FG beads (polymer magnetic nanoparticles having a particle size of about 200 nm obtained by coating ferrite particles with polyglycidyl methacrylate (poly GMA), manufactured by Tamagawa Seiki Co., Ltd.) can be suitably used.

In order to suppress adsorption of nonspecific proteins and selectively immobilize proteases, it is preferable that the nanoparticles be modified with spacer molecules capable of binding to proteases. By immobilizing proteases via spacer molecules, desorption of the proteases from the nanoparticle surface is suppressed, and position selectivity of proteolysis is improved. By adjusting the spacer molecular size, in addition, a protease is allowed to selectively access a desired position of an antibody, and position selectivity can be improved.

A spacer molecule having the above molecular diameter and capable of immobilizing a protease is preferably a non-protein, and it preferably has, at its terminus, a functional group such as an amino group, a carboxyl group, an ester group, an epoxy group, a tosyl group, a hydroxyl group, a thiol group, an aldehyde group, a maleimide group, a succinimide group, an azide group, a biotin, an avidin, or a chelate. For example, trypsin is preferably immobilized with a spacer molecule having an activated ester group. Further, a spacer arm portion other than the functional group of a spacer molecule may be a hydrophilic molecule, such as polyethylene glycol or a derivative thereof, polypropylene glycol or a derivative thereof, polyacrylamide or a derivative thereof, polyethyleneimine or a derivative thereof, poly(ethylene oxide) or a derivative thereof, or poly(ethylene terephthalic acid) or a derivative thereof.

Nanoparticles with the surfaces being modified with the spacer molecules are also commercially available, and may be used. For example, nanoparticles modified with a spacer molecule having an ester group activated with N-hydroxysuccinimide (i.e., an active ester group) is commercially available under a trade name “FG beads NHS” (Tamagawa Seiki Co., Ltd.). The particle size of FG beads NHS is about 200 nm±20 nm, and FG beads NHS is very homogeneous nanoparticles.

### <Protease>

In the nSMOL method, protease can cleave an antibody immobilized in a pore of a porous body at a specific amino acid sequence site to generate a peptide fragment containing an amino acid in the Fab region. For example, the peptide fragment can comprise an amino acid sequence containing an amino acid of the CDR2 region.

Types of proteases to be immobilized on nanoparticles may be appropriately selected in accordance with types of monoclonal antibodies to be quantified or identified via mass spectrometry. Examples of proteases include, but are not limited to, trypsin, chymotrypsin, lysyl endopeptidase, V8 protease, Asp-N protease (Asp-N), Arg-C protease (Arg-C), papain, pepsin, and dipeptidyl peptidase. Two or more types of proteases can be used in combination. As the protease, use of trypsin is particularly preferable.

When a commercially available protease is used, it is preferable to use a protease of a mass spectrometry grade or a sequencing grade. For example, trypsin of a mass spectrometry grade that has acquired improved autolysis resistance by subjecting a lysine residue of the trypsin to reductive methylation is commercially available. Depending on types of the target monoclonal antibodies, alternatively, a roughly purified protease, a protease that is not subjected to treatment to improve autolysis resistance such as reductive methylation, or a protease with trypsin activity and chymotrypsin activity may be preferably used.

Examples of proteases that can be suitably used in proteolysis in the nSMOL method of the present invention include Trypsin Gold (manufactured by Promega) and Trypsin TPCK-Treated (manufactured by Sigma).

### <Immobilization of Protease on Nanoparticle>

A method for immobilizing a protease on the nanoparticle surface is not particularly limited. An appropriate method can be adopted in accordance with characteristics of a protease and a nanoparticle (or a spacer molecule modifying the nanoparticle surface). When a protease is immobilized on the nanoparticle surface modified with a spacer, for example, a suspension of nanoparticles may be mixed with a protease-containing solution, so that the protease can be immobilized on the nanoparticle surface. Amine coupling of a nanoparticle and a protease via a functional group of the spacer molecule is preferable. For example, a carboxyl group provided on a nanoparticle via surface modification can be esterified with N-hydroxysuccinimide (NHS) to form an activated ester group, and an amino group of a protease can be bound thereto. This coupling reaction can be performed in the presence of carbodiimide as a condensing agent, and examples of carbodiimides include 1-ethyl-3-(3-dimethylaminopropyl)carbodiimide (EDAC), N,N′-dicyclohexylcarbodiimide (DCC), and bis(2,6-diisopropylphenyl)carbodiimide (DIPC). Further, an amino group of a protease may be bound to an amino group provided on a nanoparticle via surface modification using a cross-linking agent such as glutaraldehyde, bifunctional succinimide, bis(sulfosuccinimidyl)suberate (BS3), sulfonyl chloride, maleimide, or pyridyl disulfide.

The coupling method of a nanoparticle and a protease via a functional group of the spacer molecule can be performed by a simple operation of adding a protease solution to a suspension of nanoparticles and mixing and stirring the mixture under given conditions.

After the protease is immobilized on the nanoparticle surface, it is preferable to inactivate the active portion that is not bound to the protease on the nanoparticle surface. When a spacer molecule on which no protease is immobilized thereon is present on the nanoparticle surface, problems may occur. For example, an unbound spacer molecule may bind to a contaminant in the sample and adversely affect proteolysis, and a peptide fragment produced by proteolysis may be immobilized on the nanoparticle. Such problems can be suppressed by blocking the unbound spacer molecule after the protease is immobilized. As a method for inactivating the active portion unbound to the protease, chemical modification is preferable. For example, an activated ester group can be inactivated by reacting with a primary amine to form an amide bond.

Nanoparticles comprising trypsin as a protease immobilized thereon; i.e., FG beads Trypsin DART®, are included in an LC/MS/MS sample pretreatment kit “nSMOL Antibody BA Kit” (Shimadzu Corporation), and can be suitably used in the method of the present invention.

### <Proteolysis>

By contacting the porous body in which the antibody is immobilized with a nanoparticle comprising a protease immobilized on its surface in a liquid, the antibody is digested with a protease, and peptide fragments are then produced. The term “liquid” used herein refers to a situation in which a substrate (solid phase) is brought into contact with an enzyme (solid phase) in a liquid phase, and it is intended to refer to an aqueous medium suitable for a proteolysis reaction.

Proteolysis conditions are not particularly limited, and conditions similar to those for general proteolysis can be suitably adopted. For example, incubation at a temperature of about 37° C. for about 1 hour to 20 hours in a buffer solution adjusted to have a pH level in the vicinity of an optimum pH of the protease is preferable. Alternatively, incubation may be carried out under saturated vapor pressure and about 50° C. for about 3 to 8 hours.

A quantitative mixing ratio of a porous body comprising antibodies immobilized thereon with nanoparticles comprising proteases immobilized on the surfaces thereof is not particularly limited, and it may be set so as to adjust the amount of the proteases in accordance with the amount of the antibody. Under general proteolysis conditions, the substrate:protease ratio is about 100:1 to 20:1 (weight ratio). In the present invention, in contrast, access between the antibody and the protease is physically restricted by the combination of the porous body and the nanoparticles. Accordingly, it is preferable that a larger amount of proteases be used in the present invention, compared with that used for general proteolysis. For example, the antibody:protease ratio is preferably about 30:1 to 3:1, more preferably about 15:1 to 4:1, and even more preferably about 10:1 to 5:1.

More specifically, for example, the C-terminal side of an antibody is immobilized on a Protein G resin having a pore size of 100 nm, and a variable region of the antibody is always oriented to a solution side. Next, a protease is immobilized on the nanoparticle surface having a particle size of 200 nm.

The proteolysis is not particularly limited, and it can be performed under tapping rotation accompanied by periodic tapping with stirring by gentle rotation, so that the porous body and nanoparticles are homogeneously dispersed in a liquid. The term “gentle rotation” refers to, for example, a rotation speed of about 3 to 10 rpm, and the term “tapping” refers to a momentary action such as flipping or imparting a shock (e.g., a frequency of 1 to 5 actions, and preferably 2 to 4 actions, per minute). Thus, the porous body in which antibodies are immobilized is effectively brought into contact with the nanoparticles on which proteases are immobilized while maintaining a dispersed state, and proteolysis efficiency can be enhanced.

According to the method of the present invention, as described above, contact between monoclonal antibodies as substrates and proteases is restricted. Thus, a peptide derived from the Fab region exhibiting monoclonal antibody specificity can be readily and efficiently obtained by proteolysis and subjected to mass spectrometry.

### <Removal of Porous Body and Nanoparticles>

In order to subject a target peptide fragment obtained by proteolysis to mass spectrometry, it is necessary to remove the porous body and the nanoparticles. This can be achieved by subjecting the sample after proteolysis to filtration, centrifugation, magnetic separation, dialysis, and the like.

When the porous body and the nanoparticles are removed by filtration, a pore size of a filtration membrane to be used is selected in a manner such that the porous body and the nanoparticles cannot pass through the membrane but the digested peptide can pass therethrough. For example, the porous body and the nanoparticles can be easily removed by filtration using a filtration membrane made of polyvinylidene fluoride (PVDF) (low-binding hydrophilic PVDF, pore diameter: 0.2 μm, manufactured by Millipore Corporation) or a filtration membrane made of polytetrafluoroethylene (PTFE) (low-binding hydrophilic PTFE, pore diameter: 0.2 μm, manufactured by Millipore Corporation). When a means of centrifugal filtration is adopted, filtration can be quickly and easily performed.

### <Liquid Chromatography-Mass Spectrometry (LC-MS)>

By analyzing a sample containing the peptide fragment obtained above via LC-MS, antibodies can be identified and quantified.

In order to more reliably separate the peptide fragment and improve analysis accuracy, a sample before mass spectrometry may be subjected to separation and concentration via liquid chromatography (LC). When sample separation is performed via LC, an eluate from LC may be directly ionized and subjected to mass spectrometry. Analysis can also be performed via LC/MS/MS or LC/MSn that performs LC in combination with tandem mass spectrometry. Further, the eluate from LC may be collected once and then subjected to mass spectrometry. An LC column is not particularly limited, and a hydrophobic column such as C30, C18, C8, and C4 generally used in peptide analysis, a carrier for hydrophilic affinity chromatography, and the like can be appropriately selected and used.

Mass spectrometry can determine an amino acid sequence. Accordingly, whether or not a peptide fragment is derived from a specific protein such as an antibody can be determined. Based on peak intensity, in addition, concentration of a peptide fragment in a sample can be determined. At the time of analysis, a sample may be beforehand subjected to treatment, such as desalting, solubilization, extraction, concentration, or drying, if necessary.

An ionization method in mass spectrometry is not particularly limited, and an electron ionization (EI) method, a chemical ionization (CI) method, a field desorption (FD) method, a fast atom collision (FAB) method, a matrix assisted laser desorption ionization (MALDI) method, an electrospray ionization (ESI) method, and the like can be adopted. Also, a method for analyzing an ionized sample is not particularly limited, and a method of a magnetic field deflection type, a quadrupole (Q) type, an ion trap (IT) type, a time of flight (TOF) type, a Fourier transform ion cyclotron resonance (FT-ICR) type, or the like can be appropriately determined in accordance with the ionization method. Further, MS/MS analysis or multistage mass spectrometry of MS3 or higher can also be performed using triple quadrupole mass spectrometer or the like.

In recent years, a hybrid mass spectrometer referred to as a triple quadrupole has mainly been used. In this type of apparatus, an ionized biomolecule first passes through a portion referred to as an octopole, thereby reducing its ion molecular vibration radius. In a first quadrupole, subsequently, an ion having a specific mass number is selected by causing the ion to resonate, and other ions are excluded. The selected ion is brought to a second quadrupole, and cleavage is performed by colliding with argon. This reaction is referred to as collision-induced dissociation (CID). As a result of this cleavage reaction, a generated specific fragment is selected at a third quadrupole, and highly sensitive and highly selective quantification can thus be performed. This series of analyses is referred to as multiple reaction monitoring (MRM).

A device that is particularly suitably used in the method of the present invention is not particularly limited. Examples of devices include LCMS-8030, LCMS-8040, LCMS-8050, LCMS-8060, LCMS-8080, LCMS-IT-TOF, and LCMS-Q-TOF (Shimadzu Corporation).

An existing database can also be used in order to identify an antibody, based on the results of mass spectrometry. Based on the spectral information obtained by mass spectrometry, for example, putative parent ions and fragment ion series are automatically identified via Mascot search (Matrix Science). Thus, a variety of information can be obtained.

Further, it is also possible to identify an antibody by specifying the amino acid sequence of a peptide fragment via multistage mass spectrometry or other means. When a peptide fragment containing the amino acid sequence of an antibody-specific Fag region, such as CDR1, CDR2, or CDR3 region of heavy chain or light chain, can be detected, the target antibody can be identified and quantified.

When antibody identification and quantification are performed based on the results of detection, the number of amino acid residues of a peptide to be detected is preferably about 5 to 30, and more preferably about 7 to 25. When the number of amino acid residues is too small, it is difficult to distinguish the peptide to be detected from contaminants or peptide fragments derived from other sites of the same protein, and this can cause erroneous detection. When the number of amino acid residues is too large, in contrast, ionization becomes difficult, and other problems may occur. For example, detection of a peptide of interest may become difficult, or quantitative performance may deteriorate.

When antibody concentration is quantified, the amount of the antibody can be calculated based on peak area or peak intensity of a detected peptide fragment ion (in the case of multistage MS, a fragment ion obtained by cleavage of a parent ion). For example, based on a correlation between a predetermined calibration curve and a peak area or a correlation between a peak area derived from an internal standard added to a sample and a peak area derived from the sample, concentration of a peptide fragment in the sample is calculated, and the amount and the concentration of the antibody are calculated based on the concentration of the peptide fragment.

When detecting a single type of peptide via mass spectrometry, it is well known that several types of fragment ions are generated. With reference to the results of analysis of internal standard peptides and the results of analysis that are known in advance, it is possible to identify a target monoclonal antibody by detecting only one type of ion for one type of peptide; however, a plurality of fragment ions, such as two or more types, three or more types, and four or more types of fragment ions, generated from one parent ion may be simultaneously detected and quantified, so that more detailed structural information can be obtained. Because of an excessively large amount of fragment information, however, a period of analysis is prolonged, and analysis accuracy is deteriorated as a consequence. In general, accordingly, it is preferable that about 2 to 5 types of fragment ions be simultaneously monitored for one type of parent ion. While it is desirable that “y” ion series be selected for the fragment ions, “b” ion series may be selected next, in the absence of dominant candidates. Among the fragment ions, an ion having the highest ion yield is used for quantification and other ions are used for structure confirmation. Thus, structure specificity can be retained.

In order to perform simultaneous quantification of a plurality of monoclonal antibodies, antibodies are assayed within a period of a few milliseconds to a few tens of milliseconds, and continuous analyses can be performed while switching channels. Thus, a plurality of monoclonal antibodies that can be present in a sample can be quantified at a time. Detection via mass spectrometry can be carried out rapidly and accurately, and an enormous amount of information can be obtained within a short period of time. According to the method of the present invention, 2 or more, 3 or more, 4 or more, 5 or more, 10 or more, 15 or more, or 20 or more types of monoclonal antibodies can be simultaneously quantified, although the number of types of monoclonal antibodies is not limited thereto. Because of reasons such that an antibody medicine is very expensive, at present, there is substantially no conditions that 5 or more types of antibody drugs are administered to a patient in clinical settings. Accordingly, it may be possible that the method of the present invention enables simultaneous quantification of antibodies that had been administered in the past and antibodies that are currently administered to a particular patient or subject.

An LC/MS/MS sample pretreatment kit “nSMOL Antibody BA Kit” (Shimadzu Corporation) is commercialized to implement the nSMOL method. With the use of such kit in combination with LCMS-8050/8060, quantification of monoclonal antibodies can be readily performed with high accuracy at low cost.

### <Examination of Analytical Conditions>

Amino acid sequence information and the like of monoclonal antibodies to be used as antibody drugs are published, and information concerning amino acid sequences of a heavy chain and a light chain, Fab and Fc domains, a complementarity determining region (CDR), a disulfide bond, and the like can be obtained. Accordingly, a plurality of peptides can be obtained via proteolysis according to the nSMOL method. If amino acid sequence information concerning such peptides can be obtained, the positions of such peptides in the monoclonal antibodies can be easily understood. Among a plurality of peptides derived from the Fab region, accordingly, particularly preferable peptides can be selected as analytes. The peptides thus selected are referred to as “signature peptides.”

Monoclonal antibodies comprise the amino acid sequence identical or similar to that of an antibody that is endogenous to a human patient, especially in a constant region. In order to perform specific quantification, accordingly, a method of performing Fab-region-selective proteolysis to obtain a peptide is preferable. However, it should be noted that a peptide derived from the Fab region may comprise an amino acid sequence identical or similar to that of the endogenous antibody or another monoclonal antibody, which is an antibody medicine that can be present in the same sample, which is not suitable for detection.

Accordingly, it is preferable to select a signature peptide suitable for specific detection by subjecting an amino acid sequence of an analyte monoclonal antibody to alignment with amino acid sequences of other monoclonal antibodies that can be present in the same sample, as usually practiced in the art.

For sequence alignment, for example, ClustalW (http://www.ebi.ac.uk/Tools/msa/clustalw2/) provided by European Bioinformatics Institute (EBI) and available via the internet can be used. CDRs of monoclonal antibodies are deduced with the use of ClustalW, and information concerning peptides deduced to comprise the CDR sequences in at least parts thereof and to be obtained via proteolysis can be obtained.

Analytical parameters, such as signature peptides and transition, can be optimized on the basis of the obtained sequence information, with the use of Skyline developed by the group of MacCoss et al. at the University of Washington, U.S.A. (https://skyline.gs.washington.edu). In addition, LabSolutions (Shimadzu Corporation) is a system for data control, analysis, and management. By importing the obtained information thereinto, information concerning optimal MRM analytical conditions can be obtained.

By performing proteolysis by the nSMOL method in practice and using the database and the system as described above, optimal signature peptides and MRM analytical conditions for monoclonal antibodies can be more easily obtained. If optimal signature peptides and optimal MRM analytical conditions are obtained, calibration curves that can be used for monoclonal antibody quantification can be prepared in advance. Since similar validation can be attained via combined quantification of a plurality of monoclonal antibodies, a plurality of calibration curves, that can be used for simultaneous quantification of a plurality of monoclonal antibodies, can be prepared.

In cancer treatment, the site and the conditions of the disease vary among patients, and antibody drugs to be used are accordingly different. If calibration curves corresponding to a plurality of antibody drugs are simultaneously prepared in advance, accordingly, experiments for monitoring drug concentration in each specimen can be carried out, which is very effective in clinical settings.

For example, sets of calibration curves, such as a set of digestive system cancer calibration curves (e.g., Bevacizumab, Ramucirumab, Cetuximab, and Trastuzumab), a set of blood cancer calibration curves (e.g., Rituximab and Brentuximab vedotin), and a set of immunotherapy calibration curves (e.g., Nivolumab, Pembrolizumab, and Ipilimumab), may be provided. Thus, efficient dynamic information can be used for treatment.

Similarly, a set of antirheumatic drug calibration curves (e.g., Adalimumab, Infliximab, Tocilizumab, Golimumab, and Certolizumab pegol), a set of antirheumatic drug fusion protein calibration curves (e.g., Etanercept and Abatacept), and the like can be provided for antirheumatic drugs and medications for autoimmune diseases.

In addition, comprehensive field services, such as information on analytical conditions, software, a set of LCMS apparatuses, and column consumables, can be provided.

### EXAMPLES

The present invention is described in greater detail with reference to the following examples, although the technical scope of the present invention is not limited to these examples.

<Specific Procedure of the nSMOL Method>

FIG. 1 illustrates the nSMOL method employed in the present invention, and the procedure performed in the examples is described below. Reagents, containers, and the like provided by Shimadzu Corporation in the form of the “nSMOL Antibody BA Kit” together with the manufacturer's instructions may be used.

At the outset, a biological sample containing monoclonal antibodies is obtained. The biological sample is, in a clinical sense, a sample derived from blood or tissue of a patient to which monoclonal antibodies had been administered as antibody drugs, and preferably a plasma sample.

A suspension (binding solution) (25 μl) containing a porous body with a particle size of 100 nm comprising protein A which site-specifically binds to Fc domain of IgG and is immobilized in a pore (immunoglobulin collection resin, Toyopearl AF-rProtein A HC-650F resin, Tosoh Corporation) (50% suspension) and 0.1% n-octyl-β-D-thioglucopyranoside (Dojindo Laboratories) in PBS is introduced into a 2-ml tube. A plasma sample (5 μl) containing monoclonal antibodies is added thereto, and the content of the tube is mildly stirred using a vortex stirrer at 25° C. for 5 to 10 minutes.

The entire suspension is transferred to Ultrafree PVDF (0.2 μm, Merck Millipore), centrifugation is carried out at 10,000×g for 0.5 to 1 minute, and the supernatant is then removed. Subsequently, 150 μL of PBS containing 0.1% n-octyl-β-D-thioglucopyranoside (wash solution 1) is added, and centrifugation is carried out two times in the same manner as described above, followed by washing. Subsequently, 150 μL of PBS (wash solution 2) is added, and centrifugation is carried out two times in the same manner as described above, followed by washing.

After washing, the Ultrafree filter cup is transferred to a reaction vessel and pushed to the bottom. Thereafter, 80 μl of a reaction promotion solution and the internal standard (10 fmol/μL, P₁₄R) are added.

Subsequently, 10 μL of FG beads Trypsin DART® (particle size: 200 nm) (0.5 mg/ml trypsin) is added, and the reaction is carried out under saturated vapor pressure and at 50° C. with mild stirring for 4 to 6 hours.

After the reaction is terminated with the addition of 5 μL of a reaction termination solution (an aqueous solution of 10% formic acid), centrifugation is carried out at 10,000×g for 0.5 to 1 minute, the supernatant is collected in a vessel, the vessel is mounted on the magnetic stand and then allowed to stand for about 1 minute.

The supernatant is transferred to the LCMS vial and then analyzed. The supernatant contains a peptide derived from the Fab region digested via selective proteolysis according to the nSMOL method.

### <Conditions for LC-MS Analysis>

Conditions for LC-MS analysis employed in Examples are as described below.

[LC] NexeraX2 system (Shimadzu Corporation)

Columns: Shim-pack GISS C18 (50 mm×2.1 mm)

Column temperature: 50° C.

Solvent A: 0.1% formic acid/water

Solvent B: 0.1% formic acid/acetonitrile

Gradient: 1% B (1.5 minutes), 1% to 25% B (3.5 minutes), 95% B (1 minute), 1% B (1 minute)

Flow rate: 0.4 mL/minute

Amount injected: 10 μL

### [MS] LCMS-8050 and 8060 (Shimadzu Corporation)

Ionization: ESI Positive

DL temperature: 250° C.

Heat block temperature: 400° C.

Interface temperature: 300° C.

Nebulizer gas: 3 L/minute

Drying gas: 10 L/minute

Heating gas: 10 L/minute

### Example 1

Cetuximab is a human-mouse chimeric monoclonal antibody that can specifically bind to an epidermal growth factor receptor (EGFR). Amino acid sequence information of Cetuximab can be obtained from, for example, the Kyoto Encyclopedia of Genes and Genomes (KEGG). The amino acid sequences of a heavy chain and a light chain of Cetuximab are shown in SEQ ID NO: 1 and SEQ ID NO: 2, respectively.

The presence or absence of coherent peaks in plasma samples of 6 humans (3 males and 3 females) was examined. As a result, SQVFFK (SEQ ID NO: 3) in the CDR2 region of a heavy chain was selected as a peptide fragment for Cetuximab quantification. The parent ion and fragment ions of the peptide and MRM analytical conditions are shown in Table 1. One of 3 fragment ions was used for quantification and other two fragment ions were used for structure confirmation.

TABLE 1
Signature peptide and MRM analytical conditions of Cetuximab

| | | Optimal MRM conditions | | | | |
|---|---|---|---|---|---|---|
| Peptide | Region | Ion selection [m/z] | Q1 [V] | Collision [V] | Q3 [V] | Purpose |
| SQVFFK | Heavy chain CDR2 | 378.2→540.3 (y4⁺) | −17 | −15 | −28 | Quantification |
| | | 378.2→294.2 (y2⁺) | −17 | −12 | −22 | Structure confirmation |
| | | 378.2→441.2 (y3⁺) | −17 | −18 | −25 | Structure confirmation |

The method of the present invention was performed under various conditions in accordance with the guidelines of the Ministry of Health, Labour and Welfare, Japan to confirm that the method of the present invention would satisfy the standard defined by the guidelines.

Table 2 shows the results of MRM analysis performed following the nSMOL method of the plasma samples containing Cetuximab at 10 different levels from 0.586 to 300 μg/ml. As shown in Table 2, accuracy (precision) was within ±15% deviation from the theoretical value at any concentration including 0.586 μg/ml, which is the lower limit of quantification. Accordingly, it was confirmed that reproducibility would be very high when calibration curves were prepared.

TABLE 2
Reproducibility of Cetuximab quantification

| | Concentration | | | | | |
|---|---|---|---|---|---|---|
| Concentration | (quantified) (μg/ml) | | | Accuracy (%) | | |
| (theoretical) (μg/ml) | 1 | 2 | 3 | 1 | 2 | 3 |
| 0.586 | 0.567 | 0.557 | 0.513 | 96.8 | 95.0 | 87.6 |
| 1.17 | 1.31 | 1.30 | 1.22 | 112 | 111 | 104 |
| 2.34 | 2.59 | 2.64 | 2.68 | 111 | 113 | 115 |
| 4.69 | 5.19 | 4.72 | 5.33 | 111 | 101 | 114 |
| 9.38 | 9.62 | 10.2 | 10.2 | 103 | 109 | 109 |
| 18.8 | 18.9 | 19.7 | 19.9 | 100 | 105 | 106 |
| 37.5 | 37.5 | 39.4 | 35.2 | 100 | 105 | 93.9 |
| 75.0 | 72.5 | 69.2 | 70.2 | 96.7 | 92.2 | 93.6 |
| 150 | 137 | 142 | 150 | 91.7 | 94.4 | 99.7 |
| 300 | 263 | 273 | 266 | 87.7 | 90.8 | 88.6 |

Table 3 shows the results of MRM analyses of plasma samples containing Cetuximab at 4 different levels from 0.586 to 240 μg/ml performed 3 times. Each sample was subjected to measurements on different days. As shown in Table 3, accuracy was within ±15% deviation from the theoretical value at any concentration including 0.586 μg/ml, which is the lower limit of quantification, and no variation was observed in the plasma samples between immediately after preparation and those after storage.

TABLE 3
Accuracy and precision of Cetuximab quantification

| Measure- | | | | | |
|---|---|---|---|---|---|
| ment No. | Theoretical (μg/ml) | 0.586 | 1.76 | 14.1 | 240 |
| 1 | Measured (μg/ml) | 0.634 | 1.73 | 16.5 | 200 |
| | | 0.553 | 1.56 | 12.1 | 232 |
| | | 0.441 | 1.70 | 11.7 | 209 |
| | | 0.567 | 1.50 | 12.8 | 218 |
| | | 0.597 | 1.71 | 13.6 | 187 |
| | Mean (μg/ml) | 0.558 | 1.64 | 13.3 | 209 |
| | SD | 0.07 | 0.10 | 1.93 | 17.20 |
| | CV (%) | 13.0 | 6.37 | 14.5 | 8.22 |
| | Accuracy (%) | 95.3 | 93.1 | 94.9 | 87.2 |
| 2 | Measured (μg/ml) | 0.470 | 2.03 | 13.7 | 198 |
| | | 0.515 | 1.80 | 14.2 | 184 |
| | | 0.640 | 1.62 | 14.2 | 198 |
| | | 0.464 | 2.01 | 15.8 | 204 |
| | | 0.603 | 1.79 | 15.1 | 210 |
| | Mean (μg/ml) | 0.538 | 1.85 | 14.6 | 199 |
| | SD | 0.08 | 0.17 | 0.83 | 9.65 |
| | CV (%) | 14.8 | 9.25 | 5.66 | 4.28 |
| | Accuracy (%) | 91.9 | 105 | 104 | 86.3 |
| 3 | Measured (μg/ml) | 0.473 | 1.79 | 13.1 | 207 |
| | | 0.678 | 1.66 | 13.1 | 209 |
| | | 0.459 | 1.59 | 12.4 | 212 |
| | | 0.675 | 1.25 | 12.5 | 203 |
| | | 0.565 | 1.89 | 13.6 | 207 |
| | Mean (μg/ml) | 0.570 | 1.64 | 12.9 | 208 |
| | SD | 0.11 | 0.24 | 0.50 | 3.31 |
| | CV (%) | 18.5 | 14.9 | 3.87 | 1.60 |
| | Accuracy (%) | 97.3 | 93.0 | 92.0 | 86.5 |
| 1-3 | Mean (N = 15) | 0.556 | 1.71 | 13.6 | 208 |
| | SD (N = 15) | 0.08 | 0.20 | 1.37 | 10.53 |
| | CV (%) | 14.7 | 11.6 | 10.0 | 5.06 |
| | Accuracy (%) | 94.8 | 97.1 | 96.9 | 86.7 |

Table 4 shows the evaluation results of stability of plasma samples each containing Cetuximab at 1.76 μg/ml or 240 μg/ml: stability of Cetuximab in the plasma after freeze (−20° C. or −80° C.) and thaw cycles, stability thereof in the plasma after storage at room temperature for 4 hours, stability thereof in the plasma after storage at −20° C. or −80° C. for 30 days, and stability of signature peptides in a sample composition 24 or 48 hours after the pretreatment of plasma samples by the nSMOL method. As shown in Table 4, accuracy was within ±15% deviation from the theoretical value under any conditions, and the detection results were very stable after plasma samples were stored under various conditions. The results confirmed that the composition after the pretreatment by the nSMOL method was also stable.

TABLE 4
Stability of Cetuximab sample

| | Cetuximab concentration in human plasma (μg/ml) | | | |
|---|---|---|---|---|
| | 1.76 | | 240 | |
| Parameters for | Mean | Accuracy | Mean | Accuracy |
| stability evaluation | (μg/ml) | (%) | (μg/ml) | (%) |

Stability in plasma after freeze (−20° C.)/thaw cycles

| | | | | |
|---|---|---|---|---|
| Cycle 5 | 1.87 | 106 | 244 | 101 |

Stability in plasma after freeze (−80° C.)/thaw cycles

| | | | | |
|---|---|---|---|---|
| Cycle 5 | 1.75 | 99.2 | 246 | 102 |

Short-term storage stability in plasma (room temperature, 4 hours)

| | | | |
|---|---|---|---|
| 1.75 | 99.5 | 235 | 97.8 |

Long-term storage stability in plasma (−20□ C., 30 days)

| | | | |
|---|---|---|---|
| 1.86 | 105 | 215 | 89.7 |

Long-term storage stability in plasma (−80° C., 30 days)

| | | | |
|---|---|---|---|
| 1.85 | 105 | 234 | 97.7 |

Stability of sample after pretreatment in HPLC set at 5° C.

| | | | | |
|---|---|---|---|---|
| 24 hours | 1.68 | 95.2 | 214 | 89.0 |
| 48 hours | 1.75 | 99.2 | 210 | 87.3 |

Table 5 shows the results of evaluation of matrix effects of plasma samples each containing Cetuximab at 1.76 μg/ml or 240 μg/ml by employing plasma from 6 humans (3 males and 3 females) as the matrices. As shown in Table 5, a coefficient of variation (CV) for the matrix factor (MF) among individuals was 15% or lower at any concentration. It was thus confirmed that the detection results would not be influenced by individual differences in plasma compositions or other factors.

TABLE 5
Evaluation of matrix effects in Cetuximab sample

| | Concentration | | | | | |
|---|---|---|---|---|---|---|
| Analyte | (μg/ml) | Blank matrix No. | P₁₄R-normalized MF | Mean | SD | CV (%) |
| Cetuximab | 1.76 | M1 | 0.91 | 0.97 | 0.04 | 4.02 |
| | | M2 | 0.99 | | | |
| | | M3 | 0.98 | | | |
| | | F1 | 0.94 | | | |
| | | F2 | 1.02 | | | |
| | | F3 | 0.97 | | | |
| | 240 | M1 | 0.88 | 0.92 | 0.03 | 2.79 |
| | | M2 | 0.94 | | | |
| | | M3 | 0.94 | | | |
| | | F1 | 0.94 | | | |
| | | F2 | 0.90 | | | |
| | | F3 | 0.92 | | | |

Table 6 shows the results of evaluation of carry-over after measurement of the plasma samples containing Cetuximab at 300 μg/ml. In each of the measurements performed 3 times, as shown in Table 6, the peak area of the signature peptide was within 20% deviation from the results attained at the lower limit of quantification (LLOQ) and within 5% deviation from the peak area of the internal standard (P₁₄R). It was thus confirmed that the detection results would not be influenced by carry-over.

TABLE 6
Evaluation of carry-over of Cetuximab sample

| | | Peak area | | Peak area |
|---|---|---|---|---|
| Analyte | Run | LLOQ | Carry-over sample | proportion (%) |
| Cetuximab | 1 | 2855 | 170 | 5.95 |
| | 2 | 3521 | 392 | 11.1 |
| | 3 | 3123 | 409 | 13.1 |
| P₁₄R | 1 | 347412 | 8577 | 2.47 |

Table 7 shows the results of MRM analysis when the plasma samples containing Cetuximab at 500 μg/ml are diluted and analyzed. As shown in Table 7, the mean accuracy of the 10-fold diluted and 25-fold diluted samples was within ±15% deviation from the theoretical value and precision was 15% or lower. It was thus confirmed that the detection results would not be influenced by dilution of samples.

TABLE 7
Dilution integrity of Cetuximab sample

| Concentration | | Concentration | | | | |
|---|---|---|---|---|---|---|
| (theoretical) | Dilution | (measured)* | | | | Accuracy |
| (μg/ml) | factor | (μg/ml) | Mean | SD | CV (%) | (%) |
| 500 | 10 | 40.7 | 462 | 4.75 | 10.3 | 92.4 |
| | | 42.3 | | | | |
| | | 47.6 | | | | |
| | | 48.0 | | | | |
| | | 52.5 | | | | |
| 500 | 25 | 18.5 | 447 | 0.87 | 4.87 | 89.5 |
| | | 17.6 | | | | |
| | | 17.2 | | | | |
| | | 17.1 | | | | |
| | | 19.1 | | | | |

### Example 2

Rituximab is a human-mouse chimeric monoclonal antibody capable of specifically binding to CD20, which exerts therapeutic effects on B-cell non-Hodgkin's lymphoma and rheumatic arthritis. The amino acid sequences of a heavy chain and a light chain of Rituximab are shown in SEQ ID NO: 4 and SEQ ID NO: 5, respectively.

In the same manner as in Example 1, GLEWIGAIYPGNGDTSYNQK (SEQ ID NO: 6) in the CDR2 region of a heavy chain was selected as a peptide fragment for Rituximab quantification. The parent ion and the fragment ions of the peptide and MRM analytical conditions are shown in Table 8. A fragment ion was selected from 3 fragment ions and used for quantification and other two fragment ions were used for structure confirmation.

TABLE 8
Signature peptide and MRM analytical conditions of Rituximab

| | | Optimal MRM conditions | | | | |
|---|---|---|---|---|---|---|
| | | | Q1 | Collision | Q3 | |
| Peptide | Region | Ion selection [m/z] | [V] | [V] | [V] | Purpose |
| GLEWIGAIYPGNGDTSYNQK | Heavy | 1092.1→1180.6 (y11⁺) | −32 | −35 | −46 | Quantification |
| | chain | 1092.1→1343.6 (y12⁺) | −32 | −33 | −30 | Structure confirmation |
| | CDR2 | 1092.1→840.4 (b8⁺) | −32 | −33 | −24 | Structure confirmation |

Table 9 shows the results of MRM analysis performed following the nSMOL method of the plasma samples containing Rituximab at 10 different levels from 0.586 to 300 μg/ml. As shown in Table 9, accuracy (precision) was within ±15% deviation from the theoretical value at any concentration including 0.586 μg/ml, which is the lower limit of quantification.

TABLE 9
Reproducibility of Rituximab quantification

| | Concentration | | | | | |
|---|---|---|---|---|---|---|
| Concentration | (quantified) (μg/ml) | | | Accuracy (%) | | |
| (theoretical) (μg/ml) | 1 | 2 | 3 | 1 | 2 | 3 |
| 0.586 | 0.569 | 0.579 | 0.579 | 97.2 | 98.9 | 98.9 |
| 1.17 | 1.32 | 1.20 | 1.14 | 113 | 103 | 97.7 |
| 2.34 | 2.18 | 2.03 | 2.55 | 93.0 | 86.8 | 109 |
| 4.69 | 4.49 | 4.65 | 4.64 | 95.8 | 99.1 | 99.0 |
| 9.38 | 9.74 | 9.40 | 9.48 | 104 | 100 | 101 |
| 18.8 | 18.7 | 19.8 | 18.0 | 99.7 | 106 | 96.1 |
| 37.5 | 36.6 | 34.6 | 35.4 | 97.6 | 92.3 | 94.4 |
| 75.0 | 83.5 | 79.1 | 74.3 | 111 | 105 | 99.0 |
| 150 | 152 | 153 | 155 | 102 | 102 | 103 |
| 300 | 283 | 319 | 309 | 94.4 | 106 | 103 |

Table 10 shows the results of MRM analyses of plasma samples containing Rituximab at 4 different levels from 0.586 to 240 μg/ml performed 3 times. Each sample was subjected to measurements on different days. As shown in Table 10, accuracy was within ±15% deviation from the theoretical value at any concentration including 0.586 μg/ml, which is the lower limit of quantification.

TABLE 10
Accuracy and precision of Rituximab quantification

| Measure- | | | | | |
|---|---|---|---|---|---|
| ment No. | Theoretical (μg/ml) | 0.586 | 1.76 | 14.1 | 240 |
| 1 | Measured (μg/ml) | 0.578 | 1.60 | 14.4 | 247 |
| | | 0.564 | 1.56 | 15.9 | 249 |
| | | 0.610 | 1.70 | 16.9 | 264 |
| | | 0.660 | 1.83 | 15.8 | 260 |
| | | 0.498 | 1.61 | 14.7 | 253 |
| | Mean (μg/ml) | 0.582 | 1.66 | 15.5 | 255 |
| | SD | 0.06 | 0.11 | 0.99 | 7.16 |
| | CV (%) | 10.3 | 6.58 | 6.39 | 2.81 |
| | Accuracy (%) | 99.3 | 94.4 | 110 | 106 |
| 2 | Measured (μg/ml) | 0.651 | 1.70 | 15.1 | 248 |
| | | 0.648 | 2.00 | 14.8 | 268 |
| | | 0.706 | 1.94 | 14.6 | 247 |
| | | 0.606 | 1.62 | 13.3 | 225 |
| | | 0.660 | 1.87 | 14.9 | 228 |
| | Mean (μg/ml) | 0.654 | 1.83 | 14.54 | 243 |
| | SD | 0.04 | 0.16 | 0.72 | 17.72 |
| | CV (%) | 5.45 | 8.84 | 4.99 | 7.29 |
| | Accuracy (%) | 112 | 104 | 103 | 101 |
| 3 | Measured (μg/ml) | 0.590 | 2.05 | 13.6 | 256 |
| | | 0.430 | 1.79 | 14.2 | 259 |
| | | 0.611 | 2.01 | 14.4 | 245 |
| | | 0.539 | 1.98 | 13.9 | 250 |
| | | 0.563 | 1.84 | 13.7 | 244 |
| | Mean (μg/ml) | 0.547 | 1.93 | 14.0 | 251 |
| | SD | 0.07 | 0.11 | 0.36 | 6.74 |
| | CV (%) | 12.9 | 5.77 | 2.58 | 2.69 |
| | Accuracy (%) | 93.3 | 110 | 99.5 | 104 |
| 1-3 | Mean (N = 15) | 0.59 | 1.81 | 14.7 | 250 |
| | SD (N = 15) | 0.08 | 0.17 | 0.95 | 11.90 |
| | CV (%) | 11.8 | 9.22 | 6.48 | 4.77 |
| | Accuracy (%) | 101 | 103 | 104 | 104 |

Table 11 shows the evaluation results of plasma samples each containing Rituximab at 1.76 μg/ml or 240 μg/ml in terms of stability of Rituximab in the plasma after the freeze (−20° C. or −80° C.) and thaw cycles, stability thereof in the plasma after storage at room temperature for 4 hours, stability thereof in the plasma after storage at −20° C. or −80° C. for 20 days, and stability of signature peptides in a sample composition 24 or 48 hours after the pretreatment of plasma samples by the nSMOL method. As shown in Table 11, accuracy was within ±15% deviation from the theoretical value under any conditions.

TABLE 11
Stability of Rituximab sample

| | Rituximab Concentration in human plasma (μg/ml) | | | |
|---|---|---|---|---|
| | 1.76 | | 240 | |
| Parameters for | Mean | Accuracy | Mean | Accuracy |
| stability evaluation | (μg/ml) | (%) | (μg/ml) | (%) |

Stability in plasma after freeze (−20° C.)/thaw cycles

| | | | | |
|---|---|---|---|---|
| Cycle 5 | 1.91 | 108 | 235 | 97.8 |

Stability in plasma after freeze (−80° C.)/thaw cycles

| | | | | |
|---|---|---|---|---|
| Cycle 5 | 1.84 | 105 | 262 | 109 |

Short-term storage stability in plasma (room temperature, 4 hours)

| | | | |
|---|---|---|---|
| 1.68 | 95.3 | 251 | 104 |

Long-term storage stability in plasma (−20° C., 20 days)

| | | | |
|---|---|---|---|
| 1.78 | 101 | 241 | 101 |

Long-term storage stability in plasma (−80° C., 20 days)

| | | | |
|---|---|---|---|
| 1.93 | 109 | 238 | 99.3 |

Stability in sample after pretreatment in HPLC set at 5° C.

| | | | | |
|---|---|---|---|---|
| 24 hours | 1.83 | 104 | 257 | 107 |
| 48 hours | 1.77 | 101 | 239 | 99.4 |

Table 12 shows the results of evaluation of matrix effects of plasma samples each containing Rituximab at 1.76 μg/ml or 240 μg/ml by employing plasma from 6 humans (3 males and 3 females) as the matrices. As shown in Table 12, a coefficient of variation (CV) for the matrix factor (MF) among individuals was 15% or lower at any concentration.

TABLE 12
Evaluation of matrix effects in Rituximab sample

| | Con- | Blank | P₁₄R- | | | |
|---|---|---|---|---|---|---|
| | centration | matrix | normalized | | | CV |
| Analyte | (μg/ml) | No. | MF | Mean | SD | (%) |
| Rituximab | 1.76 | M1 | 2.92 | 3.06 | 0.30 | 9.87 |
| | | M2 | 2.95 | | | |
| | | M3 | 3.16 | | | |
| | | F1 | 2.91 | | | |
| | | F2 | 2.79 | | | |
| | | F3 | 3.62 | | | |
| | 240 | M1 | 0.81 | 0.85 | 0.06 | 7.34 |
| | | M2 | 0.95 | | | |
| | | M3 | 0.78 | | | |
| | | F1 | 0.83 | | | |
| | | F2 | 0.90 | | | |
| | | F3 | 0.84 | | | |

Table 13 shows the results of evaluation of carry-over after measurement of the plasma sample containing Rituximab at 300 μg/ml. In each of the measurements performed 3 times, as shown in Table 13, the peak area of the signature peptide was within 20% deviation from the results attained at the lower limit of quantification (LLOQ) and within 5% deviation from the peak area of the internal standard (P₁₄R).

TABLE 13
Evaluation of carry-over in Rituximab sample

| | | Peak area | | Peak area |
|---|---|---|---|---|
| Analyte | Run | LLOQ | Carry-over sample | proportion (%) |
| Rituximab | 1 | 2441 | 442 | 18.1 |
| | 2 | 1857 | 293 | 15.8 |
| | 3 | 2137 | 372 | 17.4 |
| P₁₄R | 1 | 289435 | 6543 | 2.3 |

Table 14 shows the results of MRM analysis when the plasma samples containing Rituximab at 500 μg/ml are diluted and analyzed. As shown in Table 14, the mean accuracy of the 10-fold diluted and 25-fold diluted samples was within ±15% deviation from the theoretical value and precision was 15% or lower.

TABLE 14
Dilution integrity of Rituximab sample

| Con- | | Con- | | | | |
|---|---|---|---|---|---|---|
| centration | | centration | | | | |
| (theoretical) | Dilution | (measured)* | | | CV | Accuracy |
| (μg/ml) | factor | (μg/ml) | Mean | SD | (%) | (%) |
| 500 | 10 | 55.1 | 556 | 1.65 | 2.97 | 111 |
| | | 55.7 | | | | |
| | | 56.7 | | | | |
| | | 57.3 | | | | |
| | | 53.1 | | | | |
| 500 | 25 | 21.6 | 521 | 0.82 | 3.94 | 104 |
| | | 20.1 | | | | |
| | | 21.5 | | | | |
| | | 19.8 | | | | |
| | | 21.1 | | | | |

### Example 3

Brentuximab vedotin is an antibody-drug complex comprising a chimeric monoclonal antibody capable of specifically binding to CD30 expressed on the surface of the cells from patients with Hodgkin's lymphoma and monomethyl auristatin E (MMAE) as a microtubule inhibitor bound thereto. The amino acid sequences of a heavy chain and a light chain of Brentuximab vedotin are shown in SEQ ID NO: 7 and SEQ ID NO: 8, respectively.

In the same manner as in Example 1, VLIYAASNLESGIPAR (SEQ ID NO: 9) in the CDR region of a light chain was selected as a peptide fragment for Brentuximab vedotin quantification. The parent ion and fragment ions of the peptide and MRM analytical conditions are shown in Table 15. A fragment ion was selected from 3 fragment ions and used for quantification and other two fragment ions were used for structure confirmation.

TABLE 15
Signature peptide and MRM analytical conditions of Brentuximab vedotin

| | | Optimal MRM conditions | | | | |
|---|---|---|---|---|---|---|
| | | | Q1 | Collision | Q3 | |
| Peptide | Region | Ion selection [m/z] | [V] | [V] | [V] | Purpose |
| VLIYAASNLESGIPAR | Light chain CDR | 837.5→343.1 (y4⁺) | −26 | −21 | −24 | Quantification |
| | | 837.5→213.1 (y2⁺) | −26 | −36 | −15 | Structure confirmation |
| | | 837.5→600.3 (y3⁺) | −26 | −32 | −22 | Structure confirmation |

Table 16 shows the results of MRM analysis performed following the nSMOL method of the plasma samples containing Brentuximab vedotin at 10 different levels from 0.586 to 300 μg/ml. As shown in Table 16, accuracy (precision) was within ±15% deviation from the theoretical value at any concentration including 0.586 μg/ml, which is the lower limit of quantification.

TABLE 16
Reproducibility of Brentuximab vedotin quantification

| | Concentration | | | | | |
|---|---|---|---|---|---|---|
| Concentration | (quantified) (μg/ml) | | | Accuracy (%) | | |
| (theoretical) (μg/ml) | 1 | 2 | 3 | 1 | 2 | 3 |
| 0.586 | 0.546 | 0.595 | 0.646 | 94.1 | 103 | 111 |
| 1.17 | 1.35 | 1.11 | 1.01 | 115 | 94.7 | 86.0 |
| 2.34 | 2.34 | 2.63 | 2.49 | 99.8 | 112 | 106 |
| 4.69 | 4.48 | 4.14 | 4.90 | 95.7 | 88.5 | 105 |
| 9.38 | 9.27 | 9.35 | 9.07 | 99.0 | 99.7 | 96.8 |
| 18.8 | 19.2 | 19.0 | 17.5 | 103 | 101 | 93.2 |
| 37.5 | 40.8 | 39.2 | 37.7 | 109 | 104 | 100 |
| 75.0 | 79.1 | 78.8 | 74.7 | 105 | 105 | 99.6 |
| 150 | 141 | 151 | 152 | 94.0 | 101 | 101 |
| 300 | 280 | 295 | 332 | 93.5 | 98.3 | 111 |

Table 17 shows the results of MRM analyses of plasma samples containing Brentuximab vedotin at 4 different levels from 0.586 to 240 μg/ml performed 3 times. Each sample was subjected to measurements on different days. As shown in Table 17, accuracy was within ±15% deviation from the theoretical value at any concentration including 0.586 μg/ml, which is the lower limit of quantification.

TABLE 17
Accuracy and precision of Brentuximab vedotin quantification

| Measure- | | | | | |
|---|---|---|---|---|---|
| ment No. | Theoretical (μg/ml) | 0.586 | 1.76 | 14.1 | 240 |
| 1 | Measured (μg/ml) | 0.626 | 1.94 | 14.2 | 243 |
| | | 0.564 | 1.71 | 14.7 | 247 |
| | | 0.512 | 1.86 | 14.3 | 259 |
| | | 0.629 | 1.70 | 14.4 | 240 |
| | | 0.657 | 1.72 | 14.0 | 242 |
| | Mean (μg/ml) | 0.598 | 1.79 | 14.3 | 246 |
| | SD | 0.06 | 0.11 | 0.26 | 7.60 |
| | CV (%) | 9.82 | 6.05 | 1.81 | 3.09 |
| | Accuracy (%) | 102 | 101 | 102 | 103 |
| 2 | Measured (μg/ml) | 0.577 | 1.78 | 12.6 | 242 |
| | | 0.514 | 1.81 | 12.5 | 212 |
| | | 0.606 | 1.80 | 13.0 | 217 |
| | | 0.611 | 1.84 | 12.9 | 211 |
| | | 0.588 | 1.79 | 12.7 | 211 |
| | Mean (μg/ml) | 0.579 | 1.80 | 12.7 | 219 |
| | SD | 0.04 | 0.02 | 0.21 | 13.32 |
| | CV (%) | 6.72 | 1.28 | 1.63 | 6.09 |
| | Accuracy (%) | 98.8 | 103 | 90.6 | 91.0 |
| 3 | Measured (μg/ml) | 0.623 | 1.85 | 14.0 | 256 |
| | | 0.560 | 1.77 | 14.2 | 250 |
| | | 0.520 | 1.75 | 13.7 | 248 |
| | | 0.571 | 1.75 | 13.9 | 249 |
| | | 0.578 | 1.81 | 14.0 | 245 |
| | Mean (μg/ml) | 0.570 | 1.79 | 13.9 | 250 |
| | SD | 0.04 | 0.04 | 0.18 | 4.04 |
| | CV (%) | 6.49 | 2.43 | 1.30 | 1.62 |
| | Accuracy (%) | 98.3 | 101 | 99.3 | 104 |
| 1-3 | Mean (N = 15) | 0.582 | 1.79 | 13.7 | 238 |
| | SD (N = 15) | 0.04 | 0.06 | 0.73 | 17.28 |
| | CV (%) | 7.57 | 3.57 | 5.33 | 7.26 |
| | Accuracy (%) | 99.4 | 102 | 97.2 | 99.1 |

Table 18 shows the evaluation results of stability of plasma samples each containing Brentuximab vedotin at 1.76 μg/ml or 240 μg/ml: stability of Brentuximab vedotin in plasma after freeze (−20° C. or −80° C.) and thaw cycles, stability thereof in plasma after storage at room temperature for 4 hours, stability thereof in plasma after storage at −20° C. or −80° C. for 30 days, and stability of signature peptides in a sample composition 24 or 48 hours after the pretreatment of plasma samples by the nSMOL method. As shown in Table 18, accuracy was within ±15% deviation from the theoretical value under any conditions.

TABLE 18
Stability of Brentuximab vedotin sample

| | Brentuximab vedotin concentration in human plasma | | | |
|---|---|---|---|---|
| | (μg/ml) | | | |
| | 1.76 | | 240 | |
| Parameters for | Mean | Accuracy | Mean | Accuracy |
| stability evaluation | (μg/ml) | (%) | (μg/ml) | (%) |

Stability in plasma after freeze (−20° C.)/thaw cycles

| | | | | |
|---|---|---|---|---|
| Cycle 5 | 1.67 | 94.7 | 238 | 99.3 |

Stability in plasma after freeze (−80° C.)/thaw cycles

| | | | | |
|---|---|---|---|---|
| Cycle 5 | 1.77 | 100 | 244 | 102 |

Short-term storage stability in plasma (room temperature, 4 hours)

| | | | |
|---|---|---|---|
| 1.58 | 89.8 | 212 | 88.5 |

Long-term storage stability in plasma (−20° C., 30 days)

| | | | |
|---|---|---|---|
| 1.71 | 97.0 | 242 | 101 |

Long-term storage stability in plasma (−80° C., 30 days)

| | | | |
|---|---|---|---|
| 1.77 | 100 | 244 | 102 |

Stability of sample after pretreatment in HPLC set at 5□ C.

| | | | | |
|---|---|---|---|---|
| 24 hours | 1.78 | 101 | 243 | 101 |
| 48 hours | 1.83 | 104 | 244 | 102 |

Table 19 shows the results of evaluation of matrix effects for plasma samples each containing Brentuximab vedotin at 1.76 μg/ml or 240 μg/ml by employing plasma from 6 humans (3 males and 3 females) as the matrices. As shown in Table 19, a coefficient of variation (CV) for the matrix factor (MF) among individuals was 15% or lower at any concentration.

TABLE 19
Evaluation of matrix effects in Brentuximab vedotin sample

| | Concentration | Blank | P₁₄R- | | | |
|---|---|---|---|---|---|---|
| Analyte | (μg/ml) | matrix No. | normalized MF | Mean | SD | CV (%) |
| Brentuximab | 1.76 | M1 | 2.51 | 3.14 | 0.37 | 11.75 |
| vedotin | | M2 | 3.65 | | | |
| | | M3 | 3.23 | | | |
| | | F1 | 3.08 | | | |
| | | F2 | 3.15 | | | |
| | | F3 | 3.21 | | | |
| | 240 | M1 | 0.97 | 0.90 | 0.08 | 8.74 |
| | | M2 | 0.94 | | | |
| | | M3 | 0.78 | | | |
| | | F1 | 0.83 | | | |
| | | F2 | 0.91 | | | |
| | | F3 | 0.97 | | | |

Table 20 shows the results of evaluation of carry-over after measurement of the plasma sample containing Brentuximab vedotin at 300 μg/ml. In each of the measurements performed 3 times, as shown in Table 20, the peak area of the signature peptide was within 20% deviation from the results attained at the lower limit of quantification (LLOQ) and within 5% deviation from the results of the internal standard (P₁₄R).

TABLE 20
Evaluation of carry-over in Brentuximab vedotin sample

| | | Peak area | | |
|---|---|---|---|---|
| | | | Carry-over | Peak area |
| Analyte | Run | LLOQ | sample | proportion (%) |
| Brentuximab | 1 | 2,656 | 363 | 13.7 |
| vedotin | 2 | 3,945 | 729 | 18.5 |
| | 3 | 4,172 | 705 | 16.9 |
| P₁₄R | 1 | 155,665 | N.D. | N.D. |

Table 21 shows the results of MRM analysis when the plasma samples containing Brentuximab vedotin at 500 μg/ml are diluted and analyzed. As shown in Table 21, the mean accuracy of the 10-fold diluted and 25-fold diluted samples was within ±15% deviation from the theoretical value and precision was 15% or lower.

TABLE 21
Dilution integrity of Brentuximab vedotin sample

| Concentration | | Concentration | | | | |
|---|---|---|---|---|---|---|
| (theoretical) | Dilution | (measured)* | | | CV | Accuracy |
| (μg/ml) | factor | (μg/ml) | Mean | SD | (%) | (%) |
| 500 | 10 | 51.1 | 472 | 2.23 | 4.71 | 94.5 |
| | | 46.6 | | | | |
| | | 45.5 | | | | |
| | | 46.4 | | | | |
| | | 46.6 | | | | |
| 500 | 25 | 20.1 | 485 | 0.66 | 3.40 | 97.1 |
| | | 19.7 | | | | |
| | | 18.9 | | | | |
| | | 18.6 | | | | |
| | | 19.9 | | | | |

### Example 4

In order to perform simultaneous quantification of a plurality of types of monoclonal antibodies, standard samples each containing Cetuximab, Rituximab, and Brentuximab vedotin at 1.76 μg/ml, 14.1 μg/ml, and 240 μg/ml, respectively, in the plasma were prepared. Control samples each containing Cetuximab, Rituximab, or Brentuximab vedotin were also prepared.

The plasma samples were treated by the nSMOL method, and the signature peptides selected in Examples 1 to 3 were subjected to independent quantification or combined quantification. The analytical conditions described above were employed herein.

As a result, samples containing 3 types of peptides were found to exhibit accuracy of within 15% compared with the samples each containing one of such 3 types of peptides, as shown in Table 22.

TABLE 22
Comparison of combined quantification and independent
quantification of 3 types of monoclonal antibodies

| mAbs | Concentration (theoretical, μg/ml) | | 1.76 | 14.1 | 240 |
|---|---|---|---|---|---|
| Brentuximab vedotin | Concentration | Combined quantification 1 | 1.92 | 15.4 | 253 |
| | (measured, μg/ml) | Combined quantification 2 | 1.86 | 14.9 | 258 |
| | | Combined quantification 3 | 1.62 | 14.6 | 230 |
| | | Independent quantification 1 | 1.76 | 15.2 | 264 |
| | | Independent quantification 2 | 1.62 | 14.8 | 263 |
| | | Independent quantification 3 | 1.64 | 13.4 | 252 |
| | | Mean | 1.74 | 14.5 | 249 |
| | | SD | 0.13 | 1.18 | 17.76 |
| | | CV (%) | 7.60 | 8.15 | 7.12 |
| | | Accuracy (%) | 99.2 | 103 | 104 |
| Cetuximab | Concentration | Combined quantification 1 | 1.64 | 14.0 | 251 |
| | (measured, μg/ml) | Combined quantification 2 | 1.53 | 12.4 | 263 |
| | | Combined quantification 3 | 1.92 | 15.0 | 244 |
| | | Independent quantification 1 | 1.69 | 14.3 | 267 |
| | | Independent quantification 2 | 1.62 | 13.0 | 268 |
| | | Independent quantification 3 | 1.83 | 14.5 | 236 |
| | | Mean | 1.71 | 13.9 | 255 |
| | | SD | 0.14 | 0.98 | 14.07 |
| | | CV (%) | 8.48 | 7.05 | 5.52 |
| | | Accuracy (%) | 97.5 | 98.6 | 106 |
| Rituximab | Concentration | Combined quantification 1 | 1.85 | 13.4 | 257 |
| | (measured, μg/ml) | Combined quantification 2 | 1.68 | 14.7 | 261 |
| | | Combined quantification 3 | 1.82 | 12.7 | 225 |
| | | Independent quantification 1 | 1.70 | 15.0 | 257 |
| | | Independent quantification 2 | 1.69 | 12.4 | 259 |
| | | Independent quantification 3 | 1.86 | 13.4 | 238 |
| | | Mean | 1.77 | 13.6 | 249 |
| | | SD | 0.15 | 1.09 | 16.45 |
| | | CV (%) | 8.65 | 7.99 | 6.60 |
| | | Accuracy (%) | 101 | 96.7 | 104 |

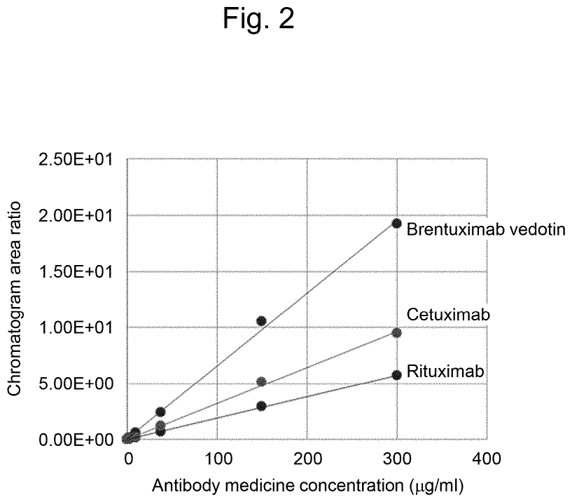

Calibration curves were prepared for the 3 types of monoclonal antibodies based on the results of quantification. As a result, results of substantially linear quantification (correlational coefficient: 0.99 or higher; calibration curve reliability: within ±15%) were obtained for all the monoclonal antibodies at the concentration from 0.586 to 300 μg/ml, as shown in FIG. 2.

Cetuximab, Rituximab, and Brentuximab vedotin are classified as chimeric antibodies, such antibodies are highly homologous to each other because they have the Fab regions of a mouse antibody, and such monoclonal antibodies have very similar structures. According to the method of the present invention, however, a biological sample containing all of the antibodies mentioned above was verified to achieve the results of quantification substantially the same as those for a sample containing only one of such monoclonal antibodies, and it was found that 3 types of monoclonal antibodies could be simultaneously quantified.

### Example 5

Ten types of monoclonal antibodies (i.e., Trastuzumab, Bevacizumab, Cetuximab, Rituximab, Nivolumab, Ipilimumab, Ramucirumab, Brentuximab vedotin, Infliximab, and Adalimumab) were added at 10 μg/ml each to the same human plasma sample, and peptides obtained by digestion by the nSMOL method were subjected to combined quantification in the same manner as in Example 4. Each of monoclonal antibodies was added at 10 μg/ml to a human plasma sample, and the resultants were independently quantified for comparison.

The sequences of peptides used for monoclonal antibody analysis and positions thereof in the antibodies are shown in Table 23. Two types of peptides were used for analysis of Rituximab and Infliximab.

TABLE 23

| Monoclonal | | |
|---|---|---|
| antibody | Peptide sequence | Position in antibody |
| Trastuzumab | IYPTNGYTR (SEQ ID NO: 10) | Heavy chain, 51-59, CDR2 |
| Bevacizumab | FTFSLDTSK (SEQ ID NO: 11) | Heavy chain, 68-76, CDR2 |
| Cetuximab | SQVFFK (SEQ ID NO: 3) | Heavy chain, 76-81, CDR2 |
| Rituximab 1 | GLEWIGAIYPGNGDTSYNQK (SEQ ID NO: 6) | Heavy chain, 44-63, CDR2 |
| Rituximab 2 | ASGYTFTSYNMHWVK (SEQ ID NO: 12) | Heavy chain, 24-38, CDR1 |
| Nivolumab | ASGITFSNSGMHWVR (SEQ ID NO: 13) | Heavy chain, 24-38, CDR1 |
| Ipilimumab | ASQSVGSSYLAWYQQKPGQAPR (SEQ ID NO: 14) | Light chain, 25-46, CDR1 |
| Ramucirumab | AFPPTFGGGTK (SEQ ID NO: 15) | Light chain, 93-103, CDR3 |
| Brentuximab | VLIYAASNLESGIPAR (SEQ ID NO: 9) | Light chain, 50-65, CDR2 |
| vedotin | | |
| Infliximab 1 | SINSATHYAESVK (SEQ ID NO: 16) | Heavy chain, 55-67, CDR2 |
| Infliximab 2 | ASQFVGSSIHWYQQR (SEQ ID NO: 17) | Light chain, 25-39, CDR1 |
| Adalimumab | APYTFGQGTK (SEQ ID NO: 18) | Light chain, 94-103, CDR3 |

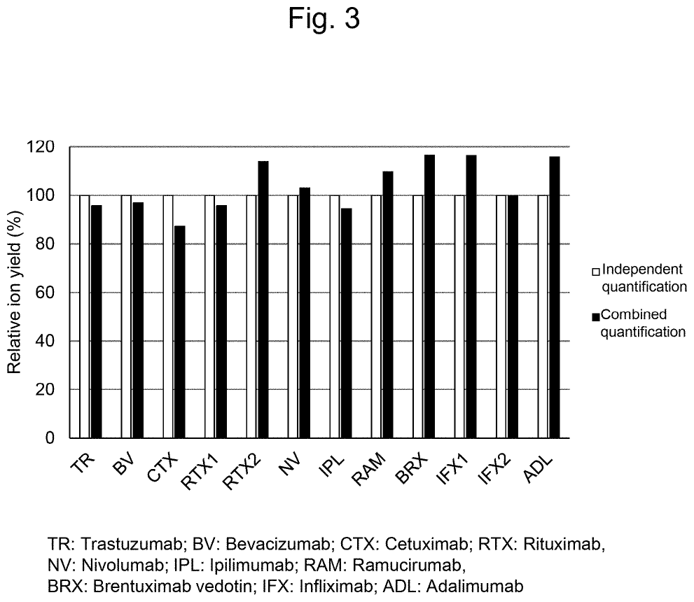

As a result, ion yields determined via combined quantification were found to be within ±20% deviation from the ion yield attained when a single type of peptide derived from each monoclonal antibody was contained, as shown in FIG. 3. Specifically, it was verified that a relative ion yield attained by independent quantification (single assay) and that attained by combined quantification (multiplex assay) are similar, and that quantification could be performed in the same manner via either single assay or multiplex assay.

### INDUSTRIAL APPLICABILITY

According to the method of the present invention, precise pharmacokinetic information concerning antibody drugs can be obtained. Accordingly, pharmacokinetic effects of antibody drugs used in combination with low-molecular-weight compounds that had not been known can be analyzed. This can open the door to clinical research concerning the influence of pharmacokinetics of antibody drugs on kinetics and drug effects of a pharmaceutical product containing a low-molecular-weight compound.

The method of the present invention accelerates reduction of analytical cost, reduction of a stress imposed on laboratory personnels, and use of drug concentration monitoring in clinical settings.

All publications, patents, and patent applications cited herein are incorporated herein by reference in their entirety.

1. A method, comprising:
bringing a porous body in which monoclonal antibodies to be measured are immobilized in pores into contact with nanoparticles on which proteases are immobilized in a liquid to perform selective proteolysis of the monoclonal antibodies;
detecting a peptide fragment including an amino acid sequence derived from the Fab region of the monoclonal antibodies via liquid chromatography-mass spectrometry (LC-MS), wherein peptide fragments of two or more types of monoclonal antibodies obtained from the same biological sample are simultaneously quantified.

2. The method according to claim 1, wherein concentration of each of the two or more types of monoclonal antibodies in a biological sample ranges between 0.5 and 300 μg/ml.

3. The method according to claim 1, wherein the results of simultaneous quantification of the two or more types of monoclonal antibodies exhibit accuracy of ±15% derivation from the results attained when each of the monoclonal antibodies are quantified independently.

4. The method according to claim 1, wherein 3, 4, 5, 6, 7, 8, 9, 10, or more types of monoclonal antibodies are simultaneously quantified.

5. The method according to claim 1, wherein the monoclonal antibodies include an antibody-drug complex.

6. The method according to claim 1, wherein the monoclonal antibodies include two or more types of antibodies selected from among: human antibodies such as Panitumumab, Ofatumumab, Golimumab, Ipilimumab, Nivolumab, Ramucirumab, and Adalimumab; humanized antibodies such as Tocilizumab, Trastuzumab, Trastuzumab-DM1, Bevacizumab, Omalizumab, Mepolizumab, Gemtuzumab, Palivizumab, Ranibizumab, Certolizumab, Ocrelizumab, Mogamulizumab, and Eculizumab; chimeric antibodies such as Rituximab, Cetuximab, Infliximab, Basiliximab, Brentuximab vedotin, and Gemtuzumab ozogamicin; and an antibody-drug complex such as Trastuzumab emtansine.

7. The method according to claim 6, wherein the monoclonal antibodies are two or three types of antibodies selected from among Cetuximab, Rituximab, and Brentuximab vedotin.

8. A composition used for combined quantification of monoclonal antibodies in a biological sample via liquid chromatography-mass spectrometry (LC-MS), which comprises two or more types of peptide fragments each comprising an amino acid sequence derived from the Fab region of a monoclonal antibody, obtained by selective proteolysis.

9. The composition according to claim 8, which yields stable results of quantification at 5° C. for 48 hours after selective proteolysis.

10. The composition according to claim 8, wherein the monoclonal antibodies include Cetuximab and a peptide fragment to be measured comprises the amino acid sequence as shown in SEQ ID NO: 3.

11. The composition according to claim 8, wherein the monoclonal antibodies include Rituximab and a peptide fragment to be measured comprises the amino acid sequence as shown in SEQ ID NO: 6.

12. The composition according to claim 8, wherein the monoclonal antibodies include Brentuximab vedotin and a peptide fragment to be measured comprises the amino acid sequence as shown in SEQ ID NO: 9.
